(19) United States
(12) Patent Application Publication
(10) Pub. No.: US 2023/0137641 A1
(43) Pub. Date: May 4, 2023

(54) METHOD FOR MEASURING HUMAN PHYSIOLOGICAL STATE AND ELECTRONIC DEVICE

(71) Applicant: Jiangyu Kangjian Innovation Medical Technology(Chengdu) Co., Ltd, Chengdu (CN)

(72) Inventors: YU LEI, Chengdu (CN); PING-HAO LIU, New Taipei (TW); ZHI-BIN HUANG, Shenzhen (CN)

(21) Appl. No.: 17/715,240

(22) Filed: Apr. 7, 2022

(30) Foreign Application Priority Data
Oct. 29, 2021 (CN) 202111272532.7

(51) Int. Cl. A61B 5/024; A61B 5/1455; A61B 5/00

(52) U.S. Cl. CPC A61B 5/02433; A61B 5/14552; A61B 5/7235; A61B 5/7225; A61B 2562/0233

(57) ABSTRACT

A method for method for measuring human pulse and blood oxygen saturation levels by irradiating with green and red light obtains a pulse wave data in a first time window. The length of a second time window is determined according to the pulse wave data and the second time window slides on the pulse wave data in the first time window. A peak position within the first time window is determined and the pulse rate is revealed according to difference in peak position and a sampling frequency of the pulse wave data. An electronic device applying such method is also disclosed.

### FIELD

The subject matter herein generally relates to field of human health, more specifically to a method for measuring physiological states and an electronic device.

### BACKGROUND

There are generally two methods to calculate pulse rate. The first is the frequency domain method, which calculates the maximum frequency point through Fourier transform. This method needs to collect a long pulse wave data to accurately obtain the pulse rate value, resulting in a long measurement time. The second is the time domain method wherein results of this method is easily affected by interference, especially when the pulse wave signal quality is poor, detection often fails or there is a false detection, resulting in low accuracy of pulse rate measurement.

Therefore, there is a room for improvement in such testing.

### BRIEF DESCRIPTION OF THE DRAWINGS

Implementations of the present disclosure will now be described, by way of embodiments, with reference to the attached figures.

FIG. 1 is flowchart of an embodiment of a method for measuring a human physiological state according to the present disclosure.

FIG. 2 is schematic diagram of an embodiment of a relationship between a first time window, a second time window, and a third time window according to the method of the present disclosure.

FIG. 3 is flowchart of another embodiment of a method according to the present disclosure.

FIG. 4 is a schematic diagram of an embodiment of pulse wave data in a frequency domain according to a method of the present disclosure.

FIG. 5 is flowchart of another embodiment of a method according to the present disclosure.

FIG. 6 is a schematic diagram of adaptive filtering of a red light signal of the method according to the present disclosure.

FIG. 7 is a schematic diagram of the effect of adaptive filtering of an infrared signal according to the method of present disclosure.

FIG. 8 is block diagram of an embodiment of an electronic device according to the present disclosure.

### DETAILED DESCRIPTION

It will be appreciated that for simplicity and clarity of illustration, where appropriate, reference numerals have been repeated among the different figures to indicate corresponding or analogous elements. Additionally, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series, and the like.

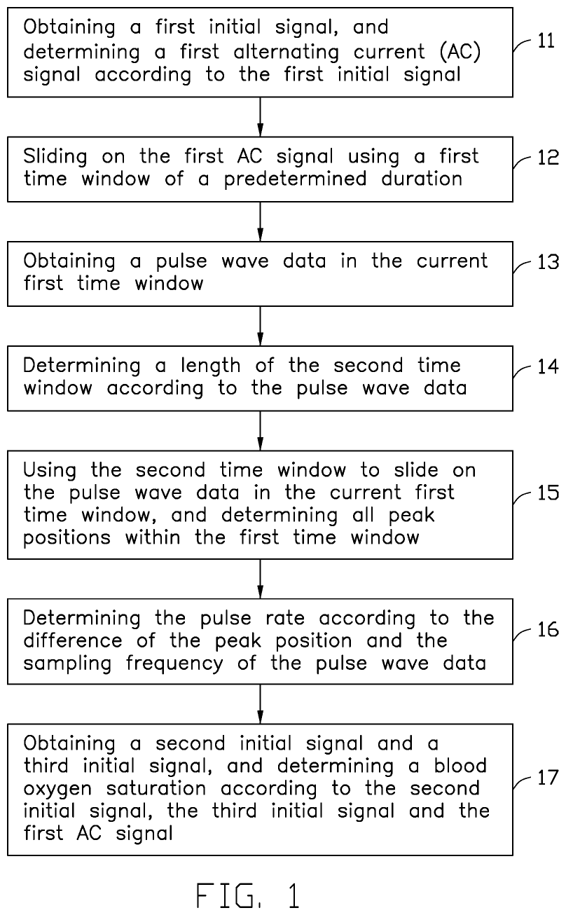

FIG. 1 shows a flow chart of one embodiment of a method for measuring physiological state of the present disclosure.

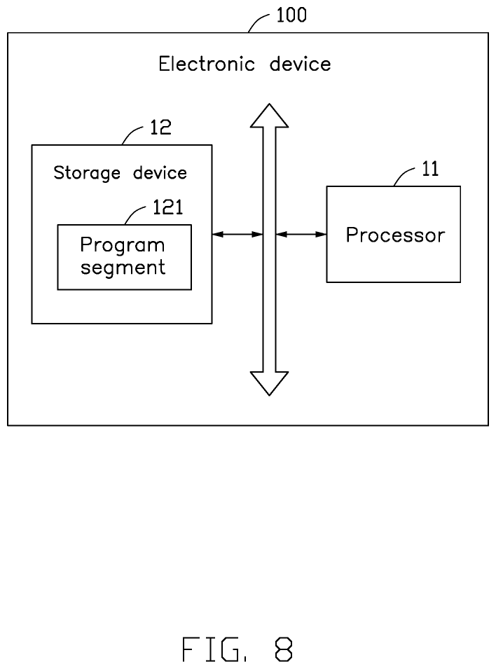

In one embodiment, the method can be applied to an electronic device 100 (shown in FIG. 8). The function for measuring provided by the method of the present disclosure can be directly integrated on the electronic device 100, or run on the electronic device 100 in the form of a software development kit (SDK).

In one embodiment, the physiological state being measured may be pulse rate.

As shown in FIG. 1, the method according to the embodiment of the present disclosure includes the following steps:

At block 11, obtaining a first initial signal, and determining a first alternating current (AC) signal according to the first initial signal.

A signal obtained by using green light as the light source for the measurement is better, and the signal-to-noise ratio is better than with other light sources. In the embodiment, green light (usually at 520 nm wavelength) as the first initial signal for measurement of pulse wave signal.

In the embodiment, determining the first AC signal according to the first initial signal includes passing the first initial signal in turn through a high pass filter and a low pass filter, to eliminate high-frequency components (such as power supply fluctuations or resonance) and low-frequency components (such as changes in capillary density and venous blood volume, temperature changes, etc.) in the pulse wave signal. The first initial signal is thereby obtained. The cut-off frequency of the high pass filter is 0.5 Hz, and the cut-off frequency of the low pass filter is 5 Hz.

The acquisition frequency of the first initial signal can be adjusted. In the embodiment, the sampling frequency of the first initial signal is 50 Hz.

At block 12, sliding on the first AC signal using a first time window of a predetermined duration.

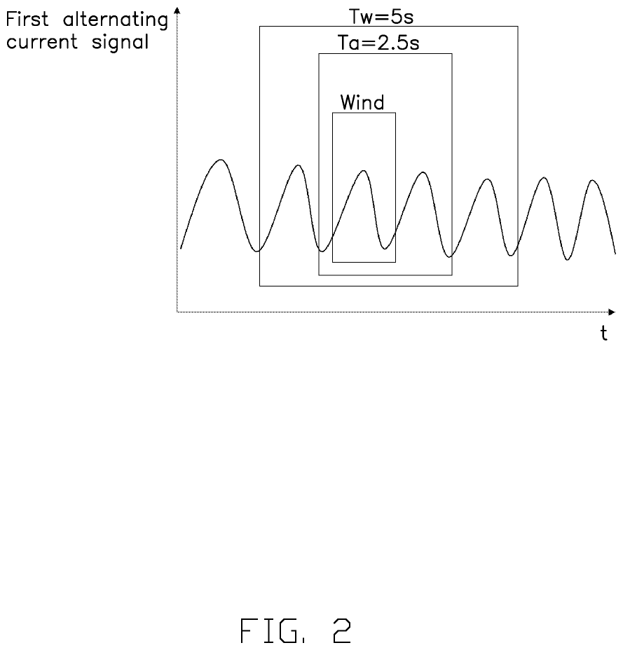

Referring to FIG. 2, the horizontal axis is time, and the vertical axis is the amplitude of the first AC signal. The predetermined duration of the first time window is adjustable, for example, the predetermined duration may be 5 seconds.

At block 13, obtaining a pulse wave data to be processed in the current first time window.

In the embodiment, the pulse wave data belongs to a part of the first AC signal.

When the first time window slides to a new position each time, only the first AC signal in the current first time window is processed. The interval of each sliding of the first time window can be set, for example, being set as the time interval between two samples.

At block 14, determining a length of the second time window according to the pulse wave data.

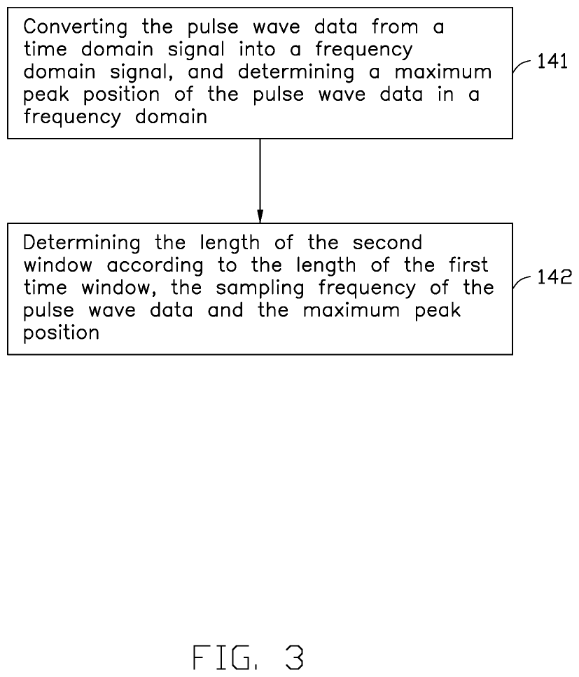

Referring to FIG. 3, block 14 according to the embodiment of the present disclosure includes the following steps:

At block 141, converting the pulse wave data from a time domain signal into a frequency domain signal, and determining a maximum peak position of the pulse wave data in a frequency domain.

In the embodiment, converting the pulse wave data from the time domain signal to the frequency domain signal may be by a Fourier transformation of the pulse wave data to determine the maximum peak position (Hamp) of the pulse wave data in the frequency domain.

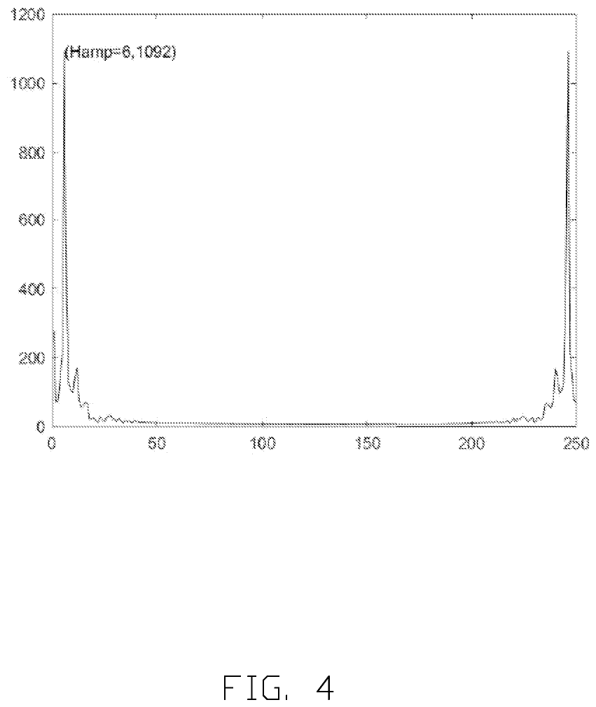

FIG. 4 shows conversion of a 5 second pulse wave data from the time domain signal to the frequency domain signal. The horizontal axis represents the sampling point, and the vertical axis represents the amplitude of the pulse wave data. As shown in FIG. 4, the maximum peak position Hamp is 6.

At block 142, determining the length of the second window according to the length of the first time window, the sampling frequency of the pulse wave data, and the maximum peak position.

Referring to FIG. 2, the duration of the second time window is determined according to the length of the first time window, the sampling frequency of the pulse wave data, and the maximum peak position. The length of the second time window will satisfy the following formula (formula (1)):

$$wind = a \times {T}_{w} \times \frac{{F}_{s}}{Hamp} \qquad ( 1 )$$

wind is the length of the second time window, a is an empirical coefficient, T_{w} is the length of the first time window, Fₛ is the sampling frequency of the pulse wave data, and Hamp is the maximum peak position.

By setting the length of the second time window dynamically, adaptive changes can be made according to the real-time pulse wave data in the first time window and the first time window. For example, when the Hamp is too large, turning down the wind, and when the Hamp is too small, turning up the wind. Therefore, the length of the second time window is suitable for different pulse rate ranges, and the accuracy of pulse rate measurement is improved.

At block 15, using the second time window to slide on the pulse wave data to be processed in the current first time window, and determining all peak positions within the first time window.

In the embodiment, determining all peak positions within the first time window includes determining whether the pulse wave data value at the center of the second time window is the maximum value in the second time window, and whether it is greater than a threshold. If the pulse wave data value at the center of the second time window is the maximum value in the second time window, this position belongs to the peak position, otherwise, sliding of the second time window is continued. For example, the second time window is slid to the right.

The interval of each sliding distance of the second time window can be set, for example, one sampling point can be set at an interval.

It can be understood that the threshold is determined according to the maximum amplitude of the pulse wave data (such as the pulse wave data in the first time window). The threshold T₁ can be obtained according to the following formula (formula (2)):

T₁=T₂×AmpMax (2)

T₁ is the threshold, T₂ is empirical coefficient, for example, T₂ is 0.6, and AmpMax is the maximum peak value of the pulse wave data in the first time window.

In some embodiments, the maximum amplitude of the pulse wave data can also be determined according to the maximum amplitude of the pulse wave data in a third time window. Each third time window is located within the first time window, and each first time window corresponds to a unique third time window, so that the standard for determination of the pulse wave peak in each first time window is consistent. The position of the third time window in the first time window can be adjusted arbitrarily, for example, it can be located on the leftmost, rightmost, or in the middle of the first time window (as shown in FIG. 2). The length Tₙ of the third time window can be 2.5 seconds. According to the monitoring range of pulse rate at the medical level, that is, 25 dpm-250 dpm, it can be seen that the peak value of a pulse wave can be collected in 0.24-2.4 seconds.

At block 16, determining the pulse rate according to the difference between the peak position and the sampling frequency of the pulse wave data.

In the embodiment, the pulse rate P can be obtained by the following formula (formula (3)).

$$P = \frac{{F}_{s} \times 60}{mRR} \qquad ( 3 )$$

Fₛ is the sampling frequency of the pulse wave data, mRR is the average difference of the peak position. The average difference of the peak position mRR can generally be taken from 5 to 8 differences of the peak position.

The embodiment of the present disclosure sets a second time window with adjustable length at the maximum peak position of the pulse wave data in the frequency domain, and uses the second time window to find the peak position of the first AC signal in the time domain. The pulse rate is finally determined from the interval of the peak position, so as to realize fast and accurate measurement of the pulse rate.

Referring to FIG. 1, the method for measuring physiological state further includes the following steps:

At block 17, obtaining a second initial signal and a third initial signal, and determining a blood oxygen saturation level according to the second initial signal, the third initial signal, and the first AC signal.

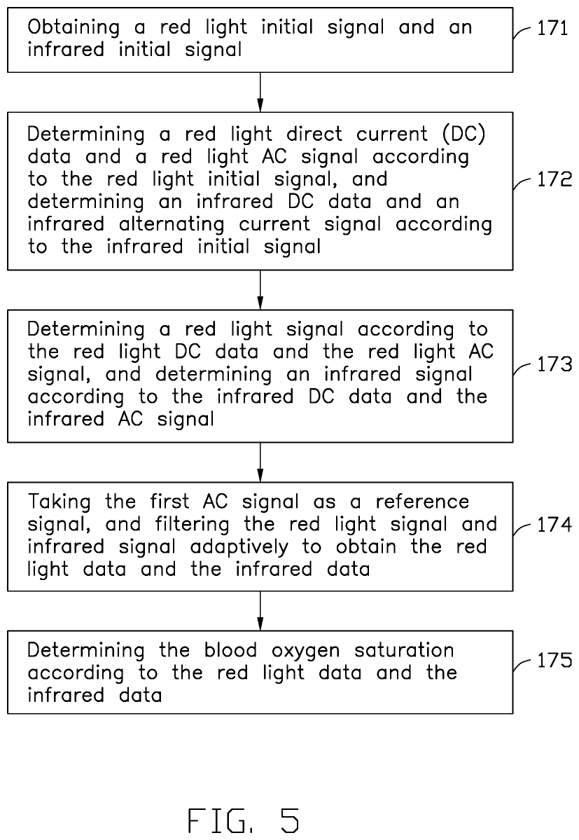

Referring to FIG. 5, the block 17 according to the embodiment of the present disclosure includes the following steps:

At block 171, obtaining a red light initial signal and an infrared initial signal.

It can be understood that in addition to irradiating the skin with the green light, the application also irradiates the skin with red light (the wavelength is usually 660 nm) and infrared light (the wavelength is usually 904 nm) for the acquisition of the red light initial signal and of the infrared initial signal.

At block 172, determining a red light direct current (DC) data and a red light AC signal according to the red light initial signal, and determining an infrared DC data and an infrared alternating current signal according to the infrared initial signal.

It can be understood that extraction of red light signal is similar to that of the green light AC signal. The AC/DC signal extraction step includes successively passing the red light initial signal and the infrared initial signal through a high pass filter and a low pass filter to eliminate the high-frequency components (such as power supply) and low-frequency components (such as changes in capillary density and venous blood volume, temperature changes, etc.) in the pulse wave signal (i.e. the red light initial signal and the infrared initial signal). In one embodiment, the cut-off frequency of the high-pass filter is 0.5 Hz, and the cut-off frequency of the low-pass filter is 5 Hz.

At block 173, determining a red light signal according to the red light DC data and the red light AC signal, and determining an infrared signal according to the infrared DC data and the infrared AC signal.

In the embodiment, determining the red light signal according to the red light DC data and the red light AC signal, and determining the infrared signal according to the infrared DC data and the infrared AC signal is as follows, the process includes dividing the red light AC data by the red light DC signal and dividing the infrared AC data by the infrared DC signal.

The red light signal N_{Rd} and the infrared signal N_{Ir} can be obtained by the following formula (4) and formula (5).

N_{Rd}=Rd_{AC}/Rd_{DC} (4)

N_{Ir}=Ir_{AC}/Ir_{DC} (5)

Rd_{AC} is the red light AC signal, Rd_{DC} is the red light DC data, Ir_{AC} is the infrared AC signal, and Ir_{DC} is the infrared DC signal.

It can be understood that the present disclosure simplifies the subsequent calculations and improves the efficiency of measuring blood oxygen saturation levels by dividing in advance the red light AC signal by the red light DC data and the infrared AC signal by the infrared DC data.

At block 174, taking the first AC signal as a reference signal, and filtering the red light signal and infrared signal adaptively to obtain the red light data and the infrared data.

The block 174 further includes comparing the red light signal with the first alternating current signal, and determining the data corresponding to at least one component signal of the red light signal as being closest to the first alternating current signal is taken as the red light data. The infrared signal is compared with the first signal, and the data corresponding to at least one component signal of the infrared signal which is closest to the first alternating current signal is taken as the infrared data.

It can be understood that the red light AC signal includes a plurality of component signals, at least one of which is an arterial signal. Other component signals may include a noise signal reflected by venous blood flow or capillaries, being less similar to the first AC signal (the AC signal of green light). On the contrary, because the green light can be better absorbed by arterial blood, the signal collected after reflection (i.e. the first AC signal) is very similar to the component containing arterial signal (i.e. infrared data) in the red light AC signal. The same is similarly true for the infrared AC signal, which will not be repeated here.

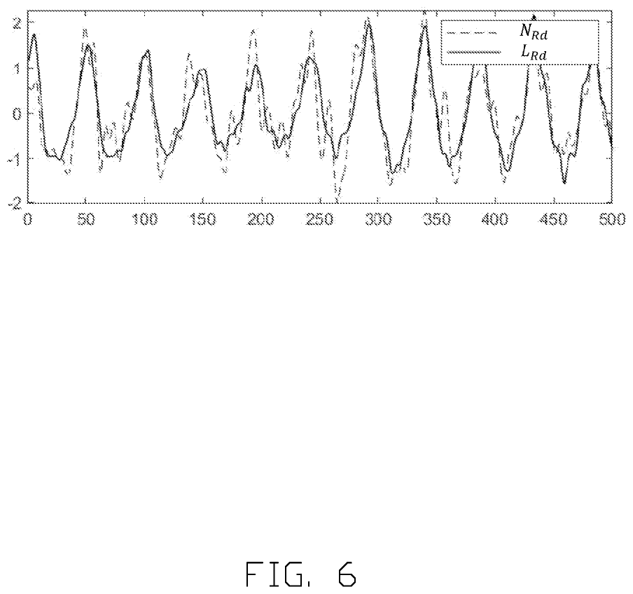

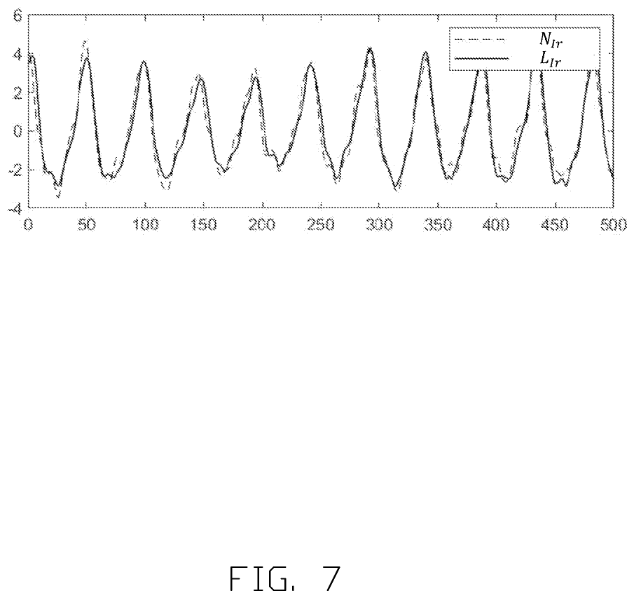

FIG. 6 shows the red light signal N_{Rd} and the red light data L_{Rd} obtained after adaptive filtering. FIG. 7 shows the infrared signal N_{Ir} and the infrared data L_{Ir} obtained after adaptive filtering.

At block 175, determining the blood oxygen saturation level according to the red light data and the infrared data.

In the embodiment, the block 175 further includes determining blood oxygen in the pulse according to the red light data and the infrared data, and determining the blood oxygen saturation level according to the pulse blood oxygen query comparison table.

The blood oxygen in the pulse can be obtained by the following formula (6):

R=L_{Rd}/L_{Ir} (6)

L_{Rd} is the red light data, and L_{Ir} is the infrared data.

In other embodiments, the blood oxygen saturation level can be determined according to the blood oxygen in the pulse. If it cannot be checked by looking up the comparison table, the blood oxygen saturation level SpO₂ can be obtained by the following formula (7):

SpO₂=A+B×R (7)

A and B are coefficients of the blood oxygen saturation level.

The embodiment of the present disclosure can adaptively filter the red light signal and the infrared signal by using the green light AC signal, effectively removing the noise signal from the red light signal and the infrared signal, and improving the accuracy in measuring the blood oxygen saturation level.

FIG. 8 illustrate an electronic device 100 in accordance with an embodiment of the present disclosure.

The electronic device 100 can further include, but is not limited to, a processor 11, a storage device 12, and a program segment 121 stored in the storage device 12. The processor 11 may execute the program code of program segment stored in the storage device 12 to implement blocks 11-17 in method shown in FIG. 1, blocks 141-142 in method shown in FIG. 3, and blocks 171-175 in method shown in FIG. 5.

It can be understood that the electronic device 100 can be, for example, a smart watch or bracelet with the function of measuring pulse rate and blood oxygen saturation.

The one or more modules may be a series of computer program instruction segments capable of completing specific functions, and the instruction segments are used to describe the execution process of the program segment 121 in the electronic device 100.

The block diagram merely shows an example of the electronic device 100 and does not constitute a limitation to the electronic device 100. In other examples, more or less components than those illustrated may be included, or some components may be combined, or different components used. For example, the electronic device 100 may also include input and output devices, network access devices, a bus, and the like.

The processor 11 may be a central processing unit (CPU), or may be other general purpose processor, a digital signal processor (DSP), an application specific integrated circuit (ASIC), a Field-Programmable gate array (FPGA) or other programmable logic device, a transistor logic device, or a discrete hardware component. The general purpose processor may be a microprocessor. The processor 11 may also be any conventional processor. The processor 11 is a control center of the electronic device 100. The processor 11 connects the parts of the electronic device 100 applying various interfaces and lines.

The storage device 12 can be used to store the program segment 121. The processor 11 operates or executes the program segment stored in the storage device 12 and recalls data stored in the storage device 12, and implements various functions of the electronic device 100. The storage device 12 mainly includes a storage program area and a storage data area, the storage program area may store an operating system, and an application (such as sound playback and image playback) required for at least one function. The storage data area may store data which is created.

The storage device 12 may include a RAM, and may also include non-volatile memory such as a hard disk, a memory, a plug-in hard disk, a smart memory card (SMC), and a Secure Digital (SD) card, a flash card, at least one disk storage device, flash device, or other volatile or non-volatile solid-state storage device.

The modules and units in the electronic device 100, if implemented in the form of software functional units and sold or used as separate products, may be stored in a computer readable storage medium. Based on such understanding, the present disclosure implements all or part of the processes in the foregoing embodiments, and the purposes of the disclosure may also be implemented and achieved by a computer program instructing related hardware. The computer program may be stored in a computer readable storage medium. The steps of the various method embodiments described above may be implemented by a computer program when executed by a processor. The computer program includes a computer program code, which may be in the form of source code, object code form, executable file, or some intermediate form. The computer readable medium may include any entity or device capable of carrying the computer program code, a recording medium, a USB flash drive, a removable hard disk, a magnetic disk, an optical disk, a computer memory, a read-only memory (ROM), a random access memory (RAM), electrical carrier signals, telecommunications signals, and software distribution media. It should be noted that the content contained in the computer readable medium can be appropriately increased or decreased according to the requirements of legislation and patent practice in jurisdictions. For example, in some jurisdictions, according to legislation and patent practice, the computer readable medium does not include electric carrier signals and telecommunication signals.

Even though numerous characteristics and advantages of the present technology have been set forth in the foregoing description, together with details of the structure and function of the present disclosure, the disclosure is illustrative only, and changes may be made in the detail, especially in matters of shape, size, and arrangement of the parts within the principles of the present disclosure, up to and including the full extent established by the broad general meaning of the terms used in the claims. It will therefore be appreciated that the exemplary embodiments described above may be modified within the scope of the claims.

What is claimed is:

1. A method of measuring physiological state configured for measuring pulse rate and comprising:
obtaining a pulse wave data in a first time window;
determining a length of a second time window according to the pulse wave data;
applying the second time window to slide on the pulse wave data in the first time window, and determining peak position within the first time window; and
determining the pulse rate according to a difference of the peak position and a sampling frequency of the pulse wave data.

2. The method according to claim 1, wherein before obtaining a pulse wave data in the first time window, the method further comprising:
obtaining a first initial signal and determining a first alternating current (AC) signal according to the first initial signal; and
sliding on the first alternating current signal by applying the first time window, and determining the first alternating current signal located in the first time window is the pulse wave data in the first time window.

3. The method according to claim 1, further comprising:
applying the second time window to slide on the pulse wave data in the first time window, and determining whether a value of the pulse wave data at center position of the second time window is a maximum value in the second time window, and is greater than a threshold value;
determining the position belongs to a peak position if the value of the pulse wave data at the center position of the second time window is a maximum value in the second time window, and greater than the threshold value; and
sliding the second time window if the value of the pulse wave data at the center position of the second time window is not a maximum value in the second time window, and is not greater than the threshold value.

4. The method according to claim 1, further comprising:
converting the pulse wave data from a time domain signal into a frequency domain signal, and determining a maximum peak position of the pulse wave data in a frequency domain; and
determining the length of the second window according to a length of the first time window, the sampling frequency of the pulse wave data and the maximum peak position.

5. The method according to claim 3, wherein after obtaining a pulse wave data in the first time window, further comprising:
obtaining the pulse wave data in a third time window, determining a maximum peak value of the peak position, and determining the threshold value according to the maximum peak value; and
wherein the third time window is located in the first time window, and each first time window corresponds to an unique third time window.

6. The method according to claim 2, wherein after determining the pulse rate according to the difference of the peak position and the sampling frequency of the pulse wave data, further comprising:
obtaining a second initial signal and a third initial signal; and
determining a blood oxygen saturation according to the second initial signal, the third initial signal and the first alternating current signal.

7. The method according to claim 6, further comprising:
obtaining a red light initial signal and an infrared initial signal, determining a red light direct current (DC) data and a red light alternating current signal according to the red light initial signal, determining an infrared direct current data and an infrared alternating current signal according to the infrared initial signal;
determining a red light signal according to the red light direct current data and the red light alternating current signal, and determining an infrared signal according to the infrared direct current data and the infrared alternating current signal;
taking the first alternating current signal as a reference signal, and filtering the red light signal and the infrared light signal adaptively to obtain a red light data and an infrared data; and
determining the blood oxygen saturation according to the red light data and the infrared data.

8. The method according to claim 7, further comprising:
comparing the red light signal with the first alternating current signal, and determining a data corresponding to at least one component signal of the red light signal closest to the first alternating current signal is the red light data; and
comparing the infrared signal with the first alternating current signal, and determining a data corresponding to at least one component signal of the infrared signal closest to the first alternating current signal is the infrared data.

9. The method according to claim 7, further comprising:
determining a pulse blood oxygen according to the red light data and the infrared data, and determining the blood oxygen saturation according to the pulse blood oxygen query comparison table.

10. An electronic device, comprising:
a storage device; and
at least one processor, wherein the storage device stores one or more programs, when executed by the at least one processor, the one or more programs cause the at least one processor to:
obtain a pulse wave data in a first time window;
determine a length of a second time window according to the pulse wave data;
apply the second time window to slide on the pulse wave data in the first time window, and determine peak position within the first time window; and
determine the pulse rate according to a difference of the peak position and a sampling frequency of the pulse wave data.

11. The electronic device according to claim 10, wherein before obtain a pulse wave data in the first time window, the at least one processor is further caused to:
obtain a first initial signal and determining a first alternating current (AC) signal according to the first initial signal; and
slide on the first alternating current signal by applying the first time window, and determine the first alternating current signal located in the first time window is the pulse wave data in the first time window.

12. The electronic device according to claim 10, wherein the at least one processor is further caused to:
apply the second time window to slide on the pulse wave data in the first time window, and determine whether a value of the pulse wave data at center position of the second time window is a maximum value in the second time window, and is greater than a threshold value;
determine the position belongs to a peak position if the value of the pulse wave data at the center position of the second time window is a maximum value in the second time window, and greater than the threshold value; and
slide the second time window if the value of the pulse wave data at the center position of the second time window is not a maximum value in the second time window, and is not greater than the threshold value.

13. The electronic device according to claim 10, wherein the at least one processor is further caused to:
convert the pulse wave data from a time domain signal into a frequency domain signal, and determine a maximum peak position of the pulse wave data in a frequency domain; and
determine the length of the second window according to a length of the first time window, the sampling frequency of the pulse wave data and the maximum peak position.

14. The electronic device according to claim 12, wherein after obtain a pulse wave data in the first time window, the at least one processor is further caused to:
obtain the pulse wave data in a third time window, determine a maximum peak value of the peak position, and determine the threshold value according to the maximum peak value; and
wherein the third time window is located in the first time window, and each first time window corresponds to an unique third time window.

15. The electronic device according to claim 11, wherein after determine the pulse rate according to the difference of the peak position and the sampling frequency of the pulse wave data, the at least one processor is further caused to:
obtain a second initial signal and a third initial signal; and
determine a blood oxygen saturation according to the second initial signal, the third initial signal and the first alternating current signal.

16. The electronic device according to claim 15, wherein the at least one processor is further caused to:
obtain a red light initial signal and an infrared initial signal, determine a red light direct current (DC) data and a red light alternating current signal according to the red light initial signal, determine an infrared direct current data and an infrared alternating current signal according to the infrared initial signal:
determine a red light signal according to the red light direct current data and the red light alternating current signal, and determine an infrared signal according to the infrared direct current data and the infrared alternating current signal;
take the first alternating current signal as a reference signal, and filter the red light signal and the infrared light signal adaptively to obtain a red light data and an infrared data; and
determine the blood oxygen saturation according to the red light data and the infrared data.

17. The electronic device according to claim 16, wherein the at least one processor is further caused to:
compare the red light signal with the first alternating current signal, and determine a data corresponding to at least one component signal of the red light signal closest to the first alternating current signal is the red light data; and
compare the infrared signal with the first alternating current signal, and determine a data corresponding to at least one component signal of the infrared signal closest to the first alternating current signal is the infrared data.

18. The electronic device according to claim 16, wherein the at least one processor is further caused to:
determine a pulse blood oxygen according to the red light data and the infrared data, and determine the blood oxygen saturation according to the pulse blood oxygen query comparison table.
